(19) United States
(12) Patent Application Publication
(10) Pub. No.: US 2019/0284162 A1
(43) Pub. Date: Sep. 19, 2019

(54) USE OF A DIFLUORO-(2-HYDROXYPROPYL)PYRIDINE COMPOUND AS A FUNGICIDE FOR CONTROL OF PHYTOPATHOGENIC FUNGI OF CORN

(71) Applicant: Dow AgroSciences LLC, Indianapolis, IN (US)

(72) Inventors: Luis Claudio Vieira da Cunha, São Paulo (BR); Courtney Gallup, Indianapolis, IN (US); Fabricio Ribeiro Baviera, São Paulo (BR)

(21) Appl. No.: 16/462,837

(22) PCT Filed: Nov. 22, 2017
(86) PCT No.: PCT/US2017/062967

§ 371 Date: May 21, 2019

Related U.S. Application Data
(60) Provisional application No. 62/425,565, filed on Nov. 22, 2016.

(51) Int. Cl. C07D 401/06; A01N 25/04; A01N 25/14; A01N 43/653

(52) U.S. Cl. CPC C07D 401/06; A01N 43/653; A01N 25/14; A01N 25/04

(57) ABSTRACT

The present disclosure is related to the field of agrochemicals, including compound I and its' use to control fungal diseases in corn. Compound (I)

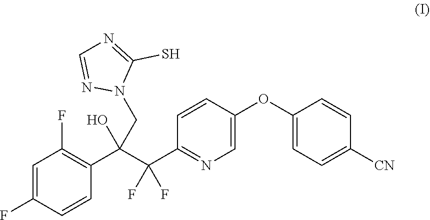 [SMILES: N#Cc1ccc(Oc2ccc(C(F)(F)C(O)(Cn3ncnc3S)c3ccc(F)cc3F)nc2)cc1]

### CROSS-REFERENCE TO RELATED APPLICATION(S)

The present application claims priority under 35 U.S.C. § 119(e) to U.S. provisional patent application, U.S. Ser. No. 62/425,565, filed Nov. 22, 2016, the entire contents of which is incorporated herein by reference.

### FIELD OF THE INVENTION

This present disclosure is related to the field of the use of 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile to control fungal diseases of corn.

### BACKGROUND AND SUMMARY OF THE INVENTION

Fungicides are compounds, of natural or synthetic origin, which act to protect and cure plants against damage caused by agriculturally-relevant fungi. Generally, no single fungicide is useful in all situations. Consequently, research is ongoing to produce fungicides that may have better performance, are easier to use, and cost less.

The present disclosure relates to 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (compound I) and its use as a fungicide. Compound I may offer protection against ascomycetes, basidiomycetes, deuteromycetes and oomycetes.

One embodiment of the present disclosure includes a method of controlling a pathogen-induced disease in a plant that is at risk of being diseased from the pathogen comprising contacting the plant or an area adjacent to the plant with a composition including compound I.

Another embodiment of the present disclosure is a use of compound I for protection of a plant against attack by a phytopathogenic organism or the treatment of a plant infested by a phytopathogenic organism, comprising the application of compound I, or a composition including compound I to soil, a plant, a part of a plant, foliage, and/or seeds.

Additionally, another embodiment of the present disclosure is a composition useful for protecting a plant against attack by a phytopathogenic organism and/or treatment of a plant infested by a phytopathogenic organism comprising compound I and a phytologically acceptable carrier material.

### DETAILED DESCRIPTION

One exemplary embodiment of the present disclosure includes mixture for controlling the growth of fungi, the mixture including compound I:

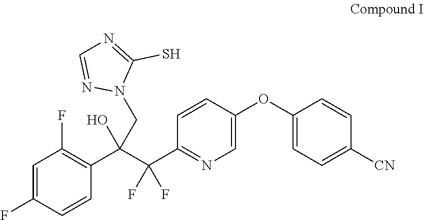 [SMILES: N#Cc1ccc(Oc2ccc(C(F)(F)C(O)(Cn3ncnc3S)c3ccc(F)cc3F)nc2)cc1]

Compound I of the present disclosure may be applied by any of a variety of known techniques, either as compound I or as formulations comprising compound I. For example, compound I may be applied to the roots, seeds or foliage of plants for the control of various fungi, without damaging the commercial value of the plants. The material may be applied in the form of any of the generally used formulation types, for example, as solutions, dusts, wettable powders, flowable concentrates, or emulsifiable concentrates.

Preferably, compound I of the present disclosure is applied in the form of a formulation, including compound I with a phytologically acceptable carrier. Concentrated formulations may be dispersed in water or other liquids for application, or formulations may be dust-like or granular, which may then be applied without further treatment. The formulations can be prepared according to procedures that are conventional in the agricultural chemical art.

The present disclosure contemplates all vehicles by which compound I may be formulated for delivery and use as a fungicide. Typically, formulations are applied as aqueous suspensions or emulsions. Such suspensions or emulsions may be produced from water-soluble, water-suspendible, or emulsifiable formulations which are solids, usually known as wettable powders; or liquids, usually known as emulsifiable concentrates, aqueous suspensions, or suspension concentrates. As will be readily appreciated, any material to which compound I may be added may be used, provided it yields the desired utility without significant interference with the activity of compound I as an antifungal agent.

Wettable powders, which may be compacted to form water-dispersible granules, comprise an intimate mixture including compound I, an inert carrier and surfactants. The concentration of compound I in the wettable powder may be from about 10 percent to about 90 percent by weight based on the total weight of the wettable powder, more preferably about 25 weight percent to about 75 weight percent. In the preparation of wettable powder formulations, compound I may be compounded with any finely divided solid, such as prophyllite, talc, chalk, gypsum, Fuller's earth, bentonite, attapulgite, starch, casein, gluten, montmorillonite clays, diatomaceous earths, purified silicates or the like. In such operations, the finely divided carrier and surfactants are typically blended with compound I and milled.

Emulsifiable concentrates of compound I may comprise a convenient concentration, such as from about 10 weight percent to about 50 weight percent of compound I, in a suitable liquid, based on the total weight of the concentrate. Compound I may be dissolved in an inert carrier, which is either a water-miscible solvent or a mixture of water-immiscible organic solvents, and emulsifiers. The concentrates may be diluted with water and oil to form spray mixtures in the form of oil-in-water emulsions. Useful organic solvents include aromatics, especially the high-boiling naphthalenic and olefinic portions of petroleum, such as heavy aromatic naphtha. Other organic solvents may also be used, for example, terpenic solvents, including rosin derivatives, aliphatic ketones, such as cyclohexanone, and complex alcohols, such as 2-ethoxyethanol.

Emulsifiers which may be advantageously employed herein may be readily determined by those skilled in the art and include various nonionic, anionic, cationic and amphoteric emulsifiers, or a blend of two or more emulsifiers. Examples of nonionic emulsifiers useful in preparing the emulsifiable concentrates include the polyalkylene glycol ethers and condensation products of alkyl and aryl phenols, aliphatic alcohols, aliphatic amines or fatty acids with ethylene oxide, propylene oxides such as the ethoxylated alkyl phenols and carboxylic esters solubilized with the polyol or polyoxyalkylene. Cationic emulsifiers include quaternary ammonium compounds and fatty amine salts. Anionic emulsifiers include the oil-soluble salts (e.g., calcium) of alkylaryl sulphonic acids, oil-soluble salts or sulfated polyglycol ethers and appropriate salts of phosphated polyglycol ether.

Representative organic liquids which may be employed in preparing the emulsifiable concentrates of compound I of the present invention are the aromatic liquids such as xylene, propyl benzene fractions; or mixed naphthalene fractions, mineral oils, substituted aromatic organic liquids such as dioctyl phthalate; kerosene; dialkyl amides of various fatty acids, particularly the dimethyl amides of fatty glycols and glycol derivatives such as the n-butyl ether, ethyl ether or methyl ether of diethylene glycol, and the methyl ether of triethylene glycol and the like. Mixtures of two or more organic liquids may also be employed in the preparation of the emulsifiable concentrate. Organic liquids include xylene, and propyl benzene fractions, with xylene being most preferred in some cases. Surface-active dispersing agents are typically employed in liquid formulations and in an amount of from 0.1 to 20 percent by weight based on the combined weight of the dispersing agent with compound I. The formulations can also contain other compatible additives, for example, plant growth regulators and other biologically active compounds used in agriculture.

Aqueous suspensions including compound I may be dispersed in an aqueous vehicle at a concentration in the range from about 5 to about 50 weight percent, based on the total weight of the aqueous suspension. Suspensions are prepared by finely grinding compound I, and vigorously mixing the ground material into a vehicle comprised of water and surfactants chosen from the same types discussed above. Other components, such as inorganic salts and synthetic or natural gums, may also be added to increase the density and viscosity of the aqueous vehicle.

Compound I may also be applied as a granular formulation, which is particularly useful for applications to the soil. Granular formulations generally contain from about 0.5 to about 10 weight percent, based on the total weight of the granular formulation of compound I, dispersed in an inert carrier which consists entirely or in large part of coarsely divided inert material such as attapulgite, bentonite, diatomite, clay or a similar inexpensive substance. Such formulations are usually prepared by dissolving compound I in a suitable solvent and applying it to a granular carrier which has been preformed to the appropriate particle size, in the range of from about 0.5 to about 3 mm. A suitable solvent is a solvent in which compound I is substantially or completely soluble. Such formulations may also be prepared by making a dough or paste of the carrier and compound I and solvent, and crushing and drying to obtain the desired granular particle.

Dusts containing compound I may be prepared by intimately mixing compound I in powdered form with a suitable dusty agricultural carrier, such as, for example, kaolin clay, ground volcanic rock, and the like. Dusts can suitably contain from about 1 to about 10 weight percent of compound I, based on the total weight of the dust.

The formulations may additionally contain adjuvant surfactants to enhance deposition, wetting and penetration of compound I onto the target crop and organism. These adjuvant surfactants may optionally be employed as a component of the formulation or as a tank mix. The amount of adjuvant surfactant will typically vary from 0.01 to 1.0 percent by volume, based on a spray-volume of water, preferably 0.05 to 0.5 volume percent. Suitable adjuvant surfactants include, but are not limited to ethoxylated nonyl phenols, ethoxylated synthetic or natural alcohols, salts of the esters or sulphosuccinic acids, ethoxylated organosilicones, ethoxylated fatty amines and blends of surfactants with mineral or vegetable oils. The formulations may also include oil-in-water emulsions such as those disclosed in U.S. patent application Ser. No. 11/495,228, the disclosure of which is expressly incorporated by reference herein.

The formulations may optionally include combinations that contain other pesticidal compounds. Such additional pesticidal compounds may be fungicides, insecticides, herbicides, nematocides, miticides, arthropodicides, bactericides or combinations thereof that are compatible with the compounds of the present invention in the medium selected for application, and not antagonistic to the activity of the present compounds. Accordingly, in such embodiments, the other pesticidal compound is employed as a supplemental toxicant for the same or for a different pesticidal use. Compound I and the pesticidal compound in the combination can generally be present in a weight ratio of from 1:100 to 100:1.

Compound I of the present disclosure may also be combined with other fungicides to form fungicidal mixtures and synergistic mixtures thereof. Compound I of the present disclosure is often applied in conjunction with one or more other fungicides to control a wider variety of undesirable diseases. When used in conjunction with other fungicide(s), the presently claimed compound I may be formulated with the other fungicide(s), tank-mixed with the other fungicide(s) or applied sequentially with the other fungicide(s). Such other fungicides may include 2-(thiocyanatomethylthio)-benzothiazole, 2-phenylphenol, 8-hydroxyquinoline sulfate, ametoctradin, amisulbrom, antimycin, Ampelomyces quisqualis, azaconazole, azoxystrobin, Bacillus subtilis, Bacillus subtilis strain QST713, benalaxyl, benomyl, benthiavalicarb-isopropyl, benzylaminobenzene-sulfonate (BABS) salt, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chlazafenone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, enestroburin, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fenpyrazamine, fentin, fentin acetate, fentin hydroxide, ferbam, ferimzone, fluazinam, fludioxonil, flumorph, fluopicolide, fluopyram, fluoroimide, fluoxastrobin, fluquinconazole, flusilazole, flusulfamide, flutianil, flutolanil, flutriafol, fluxapyroxad, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), iodocarb, ipconazole, ipfenpyrazolone, iprobenfos, iprodione, iprovalicarb, isoprothiolane, isopyrazam, isotianil, kasugamycin, kasugamycin hydrochloride hydrate, kresoxim-methyl, laminarin, mancopper, mancozeb, mandipropamid, maneb, mefenoxam, mepanipyrim, mepronil, meptyl-dinocap, mercuric chloride, mercuric oxide, mercurous chloride, metalaxyl, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pyraclostrobin, pyrametostrobin, pyraoxystrobin, pyrazophos, pyribencarb, pyributicarb, pyrifenox, pyrimethanil, pyriofenone, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griseoviridis, Trichoderma spp., (RS)-N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme, ampropylfos, anilazine, azithiram, barium polysulfide, Bayer 32394, benodanil, benquinox, bentaluron, benzamacril; benzamacril-isobutyl, benzamorf, benzovindiflupyr, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, coumoxystrobin, cufraneb, cupric hydrazinium sulfate, cuprobam, cyclafuramid, cypendazole, cyprofuram, decafentin, dichlobentiazox, dichlone, dichlozoline, diclobutrazol, dimethirimol, dinocton, dinosulfon, dinoterbon, dipyrithione, ditalimfos, dodicin, drazoxolon, EBP, enoxastrobin, ESBP, etaconazole, etem, ethirim, fenaminosulf, fenaminstrobin, fenapanil, fenitropan, fenpicoxamid, flufenoxystrobin, fluotrimazole, furcarbanil, furconazole, furconazole-cis, furmecyclox, furophanate, glyodine, griseofulvin, halacrinate, Hercules 3944, hexylthiofos, ICIA0858, ipfentrifluconazole, isofetamid, isopamphos, isovaledione, mandestrobin, mebenil, mecarbinzid, mefentriconazole, metazoxolon, methfuroxam, methylmercury dicyandiamide, metsulfovax, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, oxathiapiprolin, phenylmercury dimethyldithiocarbamate, phenylmercury nitrate, phosdiphen, prothiocarb; prothiocarb hydrochloride, pydiflumetofen, pyracarbolid, pyraziflumid, pyridinitril, pyrisoxazole, pyroxychlor, pyroxyfur, quinacetol; quinacetol sulfate, quinazamid, quinconazole, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, triclopyricarb, triflumezopyrim, urbacid, zarilamid, and any combinations thereof.

Additionally, compound I of the present invention may be combined with other pesticides, including insecticides, nematocides, miticides, arthropodicides, bactericides or combinations thereof that are compatible with compound I of the present invention in the medium selected for application, and not antagonistic to the activity of compound I, to form pesticidal mixtures and synergistic mixtures thereof. Compound I of the present disclosure may be applied in conjunction with one or more other pesticides to control a wider variety of undesirable pests. When used in conjunction with other pesticides, the presently claimed compound I may be formulated with the other pesticide(s), tank mixed with the other pesticide(s) or applied sequentially with the other pesticide(s). Typical insecticides include, but are not limited to: 1,2-dichloropropane, abamectin, acephate, acetamiprid, acethion, acetoprole, acrinathrin, acrylonitrile, alanycarb, aldicarb, aldoxycarb, aldrin, allethrin, allosamidin, allyxycarb, alpha-cypermethrin, alpha-ecdysone, alpha-endosulfan, amidithion, aminocarb, amiton, amiton oxalate, amitraz, anabasine, athidathion, azadirachtin, azamethiphos, azinphos-ethyl, azinphos-methyl, azothoate, barium hexafluorosilicate, barthrin, bendiocarb, benfuracarb, bensultap, beta-cyfluthrin, beta-cypermethrin, bifenthrin, bioallethrin, bioethanomethrin, biopermethrin, bistrifluron, borax, boric acid, bromfenvinfos, bromocyclen, bromo-DDT, bromophos, bromophos-ethyl, bufencarb, buprofezin, butacarb, butathiofos, butocarboxim, butonate, butoxycarboxim, cadusafos, calcium arsenate, calcium polysulfide, camphechlor, carbanolate, carbaryl, carbofuran, carbon disulfide, carbon tetrachloride, carbophenothion, carbosulfan, cartap, cartap hydrochloride, chlorantraniliprole, chlorbicyclen, chlordane, chlordecone, chlordimeform, chlordimeform hydrochloride, chlorethoxyfos, chlorfenapyr, chlorfenvinphos, chlorfluazuron, chlormephos, chloroform, chloropicrin, chlorphoxim, chlorprazophos, chlorpyrifos, chlorpyrifos-methyl, chlorthiophos, chromafenozide, cinerin I, cinerin II, cinerins, cismethrin, cloethocarb, closantel, clothianidin, copper acetoarsenite, copper arsenate, copper naphthenate, copper oleate, coumaphos, coumithoate, crotamiton, crotoxyphos, crufomate, cryolite, cyanofenphos, cyanophos, cyanthoate, cyantraniliprole, cyclethrin, cycloprothrin, cyfluthrin, cyhalothrin, cypermethrin, cyphenothrin, cyromazine, cythioate, DDT, decarbofuran, deltamethrin, demephion, demephion-O, demephion-S, demeton, demeton-methyl, demeton-O, demeton-O-methyl, demeton-S, demeton-S-methyl, demeton-S-methylsulphon, diafenthiuron, dialifos, diatomaceous earth, diazinon, dicapthon, dichlofenthion, dichlorvos, dicresyl, dicrotophos, dicyclanil, dieldrin, diflubenzuron, dilor, dimefluthrin, dimefox, dimetan, dimethoate, dimethrin, dimethylvinphos, dimetilan, dinex, dinex-diclexine, dinoprop, dinosam, dinotefuran, diofenolan, dioxabenzofos, dioxacarb, dioxathion, disulfoton, dithicrofos, d-limonene, DNOC, DNOC-ammonium, DNOC-potassium, DNOC-sodium, doramectin, ecdysterone, emamectin, emamectin benzoate, EMPC, empenthrin, endosulfan, endothion, endrin, EPN, epofenonane, eprinomectin, esdepalléthrine, esfenvalerate, etaphos, ethiofencarb, ethion, ethiprole, ethoate-methyl, ethoprophos, ethyl formate, ethyl-DDD, ethylene dibromide, ethylene dichloride, ethylene oxide, etofenprox, etrimfos, EXD, famphur, fenamiphos, fenazaflor, fenchlorphos, fenethacarb, fenfluthrin, fenitrothion, fenobucarb, fenoxacrim, fenoxycarb, fenpirithrin, fenpropathrin, fensulfothion, fenthion, fenthion-ethyl, fenvalerate, fipronil, flonicamid, flubendiamide, flucofuron, flucycloxuron, flucythrinate, flufenerim, flufenoxuron, flufenprox, fluvalinate, fonofos, formetanate, formetanate hydrochloride, formothion, formparanate, formparanate hydrochloride, fosmethilan, fospirate, fosthietan, furathiocarb, furethrin, gamma-cyhalothrin, gamma-HCH, halfenprox, halofenozide, HCH, HEOD, heptachlor, heptenophos, heterophos, hexaflumuron, HHDN, hydramethylnon, hydrogen cyanide, hydroprene, hyquincarb, imidacloprid, imiprothrin, indoxacarb, iodomethane, IPSP, isazofos, isobenzan, isocarbophos, isodrin, isofenphos, isofenphos-methyl, isoprocarb, isoprothiolane, isothioate, isoxathion, ivermectin, jasmolin I, jasmolin II, jodfenphos, juvenile hormone I, juvenile hormone II, juvenile hormone III, kelevan, kinoprene, lambda-cyhalothrin, lead arsenate, lepimectin, leptophos, lindane, lirimfos, lufenuron, lythidathion, malathion, malonoben, mazidox, mecarbam, mecarphon, menazon, mephosfolan, mercurous chloride, mesulfenfos, metaflumizone, methacrifos, methamidophos, methidathion, methiocarb, methocrotophos, methomyl, methoprene, methoxychlor, methoxyfenozide, methyl bromide, methyl isothiocyanate, methylchloroform, methylene chloride, metofluthrin, metolcarb, metoxadiazone, mevinphos, mexacarbate, milbemectin, milbemycin oxime, mipafox, mirex, molosultap, monocrotophos, monomehypo, monosultap, morphothion, moxidectin, naftalofos, naled, naphthalene, nicotine, nifluridide, nitenpyram, nithiazine, nitrilacarb, novaluron, noviflumuron, omethoate, oxamyl, oxydemeton-methyl, oxydeprofos, oxydisulfoton, para-dichlorobenzene, parathion, parathion-methyl, penfluron, pentachlorophenol, permethrin, phenkapton, phenothrin, phenthoate, phorate, phosalone, phosfolan, phosmet, phosnichlor, phosphamidon, phosphine, phoxim, phoxim-methyl, pirimetaphos, pirimicarb, pirimiphos-ethyl, pirimiphos-methyl, potassium arsenite, potassium thiocyanate, pp′-DDT, prallethrin, precocene I, precocene II, precocene III, primidophos, profenofos, profluralin, promacyl, promecarb, propaphos, propetamphos, propoxur, prothidathion, prothiofos, prothoate, protrifenbute, pyraclofos, pyrafluprole, pyrazophos, pyresmethrin, pyrethrin I, pyrethrin II, pyrethrins, pyridaben, pyridalyl, pyridaphenthion, pyrifluquinazon, pyrimidifen, pyrimitate, pyriprole, pyriproxyfen, quassia, quinalphos, quinalphos-methyl, quinothion, rafoxanide, resmethrin, rotenone, ryania, sabadilla, schradan, selamectin, silafluofen, silica gel, sodium arsenite, sodium fluoride, sodium hexafluorosilicate, sodium thiocyanate, sophamide, spinetoram, spinosad, spiromesifen, spirotetramat, sulcofuron, sulcofuron-sodium, sulfluramid, sulfotep, sulfoxaflor, sulfuryl fluoride, sulprofos, tau-fluvalinate, tazimcarb, TDE, tebufenozide, tebufenpyrad, tebupirimfos, teflubenzuron, tefluthrin, temephos, TEPP, terallethrin, terbufos, tetrachloroethane, tetrachlorvinphos, tetramethrin, tetramethylfluthrin, theta-cypermethrin, thiacloprid, thiamethoxam, thicrofos, thiocarboxime, thiocyclam, thiocyclam oxalate, thiodicarb, thiofanox, thiometon, thiosultap, thiosultap-disodium, thiosultap-monosodium, thuringiensin, tolfenpyrad, tralomethrin, transfluthrin, transpermethrin, triarathene, triazamate, triazophos, trichlorfon, trichlormetaphos-3, trichloronat, trifenofos, triflumuron, trimethacarb, triprene, vamidothion, vaniliprole, XMC, xylylcarb, zeta-cypermethrin, zolaprofos, and any combinations thereof.

Additionally, compound I of the present invention may be combined with herbicides that are compatible with compound I of the present invention in the medium selected for application, and not antagonistic to the activity of compound I to form pesticidal mixtures and synergistic mixtures thereof. The fungicidal compound I of the present disclosure may be applied in conjunction with one or more herbicides to control a wide variety of undesirable plants. When used in conjunction with herbicides, the presently claimed compound I may be formulated with the herbicide(s), tank mixed with the herbicide(s) or applied sequentially with the herbicide(s). Typical herbicides include, but are not limited to: 4-CPA; 4-CPB; 4-CPP; 2,4-D; 3,4-DA; 2,4-DB; 3,4-DB; 2,4-DEB; 2,4-DEP; 3,4-DP; 2,3,6-TBA; 2,4,5-T; 2,4,5-TB; acetochlor, acifluorfen, aclonifen, acrolein, alachlor, allidochlor, alloxydim, allyl alcohol, alorac, ametridione, ametryn, amibuzin, amicarbazone, amidosulfuron, aminocyclopyrachlor, aminopyralid, amiprofos-methyl, amitrole, ammonium sulfamate, anilofos, anisuron, asulam, atraton, atrazine, azafenidin, azimsulfuron, aziprotryne, barban, BCPC, beflubutamid, benazolin, bencarbazone, benfluralin, benfuresate, bensulfuron, bensulide, bentazone, benzadox, benzfendizone, benzipram, benzobicyclon, benzofenap, benzofluor, benzoylprop, benzthiazuron, bicyclopyrone, bifenox, bilanafos, bispyribac, borax, bromacil, bromobonil, bromobutide, bromofenoxim, bromoxynil, brompyrazon, butachlor, butafenacil, butamifos, butenachlor, buthidazole, buthiuron, butralin, butroxydim, buturon, butylate, cacodylic acid, cafenstrole, calcium chlorate, calcium cyanamide, cambendichlor, carbasulam, carbetamide, carboxazole chlorprocarb, carfentrazone, CDEA, CEPC, chlomethoxyfen, chloramben, chloranocryl, chlorazifop, chlorazine, chlorbromuron, chlorbufam, chloreturon, chlorfenac, chlorfenprop, chlorflurazole, chlorflurenol, chloridazon, chlorimuron, chlornitrofen, chloropon, chlorotoluron, chloroxuron, chloroxynil, chlorpropham, chlorsulfuron, chlorthal, chlorthiamid, cinidon-ethyl, cinmethylin, cinosulfuron, cisanilide, clethodim, cliodinate, clodinafop, clofop, clomazone, clomeprop, cloprop, cloproxydim, clopyralid, cloransulam, CMA, copper sulfate, CPMF, CPPC, credazine, cresol, cumyluron, cyanatryn, cyanazine, cycloate, cyclosulfamuron, cycloxydim, cycluron, cyhalofop, cyperquat, cyprazine, cyprazole, cypromid, daimuron, dalapon, dazomet, delachlor, desmedipham, desmetryn, di-allate, dicamba, dichlobenil, dichloralurea, dichlormate, dichlorprop, dichlorprop-P, diclofop, diclosulam, diethamquat, diethatyl, difenopenten, difenoxuron, difenzoquat, diflufenican, diflufenzopyr, dimefuron, dimepiperate, dimethachlor, dimethametryn, dimethenamid, dimethenamid-P, dimexano, dimidazon, dinitramine, dinofenate, dinoprop, dinosam, dinoseb, dinoterb, diphenamid, dipropetryn, diquat, disul, dithiopyr, diuron, DMPA, DNOC, DSMA, EBEP, eglinazine, endothal, epronaz, EPTC, erbon, esprocarb, ethalfluralin, ethametsulfuron, ethidimuron, ethiolate, ethofumesate, ethoxyfen, ethoxysulfuron, etinofen, etnipromid, etobenzanid, EXD, fenasulam, fenoprop, fenoxaprop, fenoxaprop-P, fenoxasulfone, fenteracol, fenthiaprop, fentrazamide, fenuron, ferrous sulfate, flamprop, flamprop-M, flazasulfuron, florasulam, fluazifop, fluazifop-P, fluazolate, flucarbazone, flucetosulfuron, fluchloralin, flufenacet, flufenican, flufenpyr, flumetsulam, flumezin, flumiclorac, flumioxazin, flumipropyn, fluometuron, fluorodifen, fluoroglycofen, fluoromidine, fluoronitrofen, fluothiuron, flupoxam, flupropacil, flupropanate, flupyrsulfuron, fluridone, flurochloridone, fluroxypyr, flurtamone, fluthiacet, fomesafen, foramsulfuron, fosamine, furyloxyfen, glufosinate, glufosinate-P, glyphosate, halosafen, halosulfuron, haloxydine, haloxyfop, haloxyfop-P, hexachloroacetone, hexaflurate, hexazinone, imazamethabenz, imazamox, imazapic, imazapyr, imazaquin, imazethapyr, imazosulfuron, indanofan, indaziflam, iodobonil, iodomethane, iodosulfuron, ioxynil, ipazine, ipfencarbazone, iprymidam, isocarbamid, isocil, isomethiozin, isonoruron, isopolinate, isopropalin, isoproturon, isouron, isoxaben, isoxachlortole, isoxaflutole, isoxapyrifop, karbutilate, ketospiradox, lactofen, lenacil, linuron, MAA, MAMA, MCPA, MCPA-thioethyl, MCPB, mecoprop, mecoprop-P, medinoterb, mefenacet, mefluidide, mesoprazine, mesosulfuron, mesotrione, metam, metamifop, metamitron, metazachlor, metazosulfuron, metflurazon, methabenzthiazuron, methalpropalin, methazole, methiobencarb, methiozolin, methiuron, methometon, methoprotryne, methyl bromide, methyl isothiocyanate, methyldymron, metobenzuron, metobromuron, metolachlor, metosulam, metoxuron, metribuzin, metsulfuron, molinate, monalide, monisouron, monochloroacetic acid, monolinuron, monuron, morfamquat, MSMA, naproanilide, napropamide, naptalam, neburon, nicosulfuron, nipyraclofen, nitralin, nitrofen, nitrofluorfen, norflurazon, noruron, OCH, orbencarb, ortho-dichlorobenzene, orthosulfamuron, oryzalin, oxadiargyl, oxadiazon, oxapyrazon, oxasulfuron, oxaziclomefone, oxyfluorfen, parafluron, paraquat, pebulate, pelargonic acid, pendimethalin, penoxsulam, pentachlorophenol, pentanochlor, pentoxazone, perfluidone, pethoxamid, phenisopham, phenmedipham, phenmedipham-ethyl, phenobenzuron, phenylmercury acetate, picloram, picolinafen, pinoxaden, piperophos, potassium arsenite, potassium azide, potassium cyanate, pretilachlor, primisulfuron, procyazine, prodiamine, profluazol, profluralin, profoxydim, proglinazine, prometon, prometryn, propachlor, propanil, propaquizafop, propazine, propham, propisochlor, propoxycarbazone, propyrisulfuron, propyzamide, prosulfalin, prosulfocarb, prosulfuron, proxan, prynachlor, pydanon, pyraclonil, pyraflufen, pyrasulfotole, pyrazolynate, pyrazosulfuron, pyrazoxyfen, pyribenzoxim, pyributicarb, pyriclor, pyridafol, pyridate, pyriftalid, pyriminobac, pyrimisulfan, pyrithiobac, pyroxasulfone, pyroxsulam, quinclorac, quinmerac, quinoclamine, quinonamid, quizalofop, quizalofop-P, rhodethanil, rimsulfuron, saflufenacil, S-metolachlor, sebuthylazine, secbumeton, sethoxydim, siduron, simazine, simeton, simetryn, SMA, sodium arsenite, sodium azide, sodium chlorate, sulcotrione, sulfallate, sulfentrazone, sulfometuron, sulfosulfuron, sulfuric acid, sulglycapin, swep, TCA, tebutam, tebuthiuron, tefuryltrione, tembotrione, tepraloxydim, terbacil, terbucarb, terbuchlor, terbumeton, terbuthylazine, terbutryn, tetrafluron, thenylchlor, thiazafluron, thiazopyr, thidiazimin, thidiazuron, thiencarbazone-methyl, thifensulfuron, thiobencarb, tiocarbazil, tioclorim, topramezone, tralkoxydim, triafamone, tri-allate, triasulfuron, triaziflam, tribenuron, tricamba, triclopyr, tridiphane, trietazine, trifloxysulfuron, trifluralin, triflusulfuron, trifop, trifopsime, trihydroxytriazine, trimeturon, tripropindan, tritac, tritosulfuron, vernolate, and xylachlor.

One embodiment of the present disclosure is a method for the control or prevention of fungal attack. This method comprises applying to the soil, plant, roots, foliage, seed or locus of the fungus, or to a locus in which the infestation is to be prevented (for example applying to cereal or grape plants), a fungicidal effective amount of compound I. Compound I is suitable for treatment of various plants at fungicidal levels, while exhibiting low phytotoxicity. Compound I may be useful both in a protectant and/or an eradicant fashion.

Compound I has been found to have significant fungicidal effects on phytopathogenic fungi of corn. These diseases include, but are not limited to, Puccinia polysora, which causes brown rust of corn; Cercospora zeae-maydis, which causes grey leaf spot of corn; Phaeosphaeria maydis, which causes phaeosphaeria leaf spot of corn; and Exserohilum turcicum, which causes northern corn leaf blight; particularly for agricultural use. Compound I is particularly effective for use with agricultural crops and horticultural plants.

It will be understood by those in the art that the efficacy of the compound for the foregoing fungi establishes the general utility of compound I as a fungicide. In particular, the composition is effective in controlling a variety of undesirable fungi that infect useful plant crops. The composition maybe used against a variety of Ascomycete and Basidiomycete fungi, including for example the following representative fungi species: Anthracnose (Colletotrichum graminicola, Glomerella tucumanensis), Aspergillus ear and kernel rot (Aspergillus flavus), banded leaf and sheath spot (Rhizoctonia solani, Rhizoctonia microsclerotia), black bundle disease (Acremonium strictum, Cephalosporium acremonium), black kernel rot (Marasmiellus spp.), brown spot, black spot, stalk rot (Physoderma maydis), Cephalosporium kernel rot (Acremonium strictum, Cephalosporium acremonium), charcoal rot (Macrophomina phaseolina), Corticium ear rot (Thanatephorus cucumeris, Corticium sasakii), Curvularia leaf spot (Curvularia spp.), Didymella leaf spot (Didymella exitalis), Diplodia ear rot, stalk rot, leaf spot, leaf streak (Diplodia spp.), dry ear rot, cob, kernel and stalk rot (Nigrospora oryzae) ear rots (Alternaria spp., Aspergillus spp., Botrytis cinerea, Fusarium culmorum, Cunninghamella spp., Curvularia pallescens, Rhizopus spp., Gonatobotrys simplex, Doratomyces stemonitis, Cephalotrichum stemonitis, Pithomyces maydicus, Scopulariopsis brumptii), ergot (Claviceps gigantea), eyespot (Aureobasidium zeae, Kabatiella zeae), Fusarium kernel, root and stalk rot, seed rot, stalk rot, ear rot, and seedling blight (Fusarium spp., Gibberella spp.), gray ear rot (Botryosphaeria zeae, Physalospora zeae), gray leaf spot, Cercospora leaf spot (Cercospora sorghi, Cercospora zeae-maydis), Helminthosporium root rot (Exserohilum pedicellatum, Helminthosporium pedicellatum), Cladosporium rot (Cladosporium spp.), leaf spots (Alternaria alternata, Ascochyta maydis, Ascochyta tritici, Ascochyta zeicola, Bipolaris victoriae, Helminthosporium victoriae, Cochliobolus victoriae, Cochliobolus sativus, Bipolaris sorokiniana, Epicoccum nigrum, Exserohilum prolatum, Drechslera prolata, Graphium penicillioides, Leptosphaeria maydis, Leptothyrium zeae, Ophiosphaerella herpotricha, Paraphaeosphaeria michotii, Phoma spp., Septoria spp.), Northern corn leaf blight (Setosphaeria turcica), white blast, stalk rot, stripe (Exserohilum turcicum, Helminthosporium turcicum), Northern corn leaf spot (Cochliobolus carbonum), Helminthosporium ear rot (Bipolaris zeicola, Helminthosporium carbonum), Penicillium ear rot, blue eye, blue mold (Pennicillium spp.), Phaeocytostroma stalk rot and root rot (Phaeocytostroma ambiguum, Phaeocytosporella zeae), Phaeosphaeria leaf spot (Phaeosphaeria maydis, Sphaerulina maydis), Physalospora ear rot, Botryosphaeria ear rot (Botryosphaeria festucae, Physalospora zeicola), Pyrenochaeta stalk rot and root rot (Phoma terrestris, Pyrenochaeta terrestris), red kernel disease, ear mold, leaf and seed rot (Epicoccum nigrum), Rhizoctonia ear rot, root rot, stalk rot (Rhizoctonia spp.), root rots (Alternaria alternata, Cercospora sorghi, Dictochaeta fertilis, Fusarium spp., Gibberella spp., Microdochium bolleyi, Mucor sp., Periconia circinata, Rhizopus arrhizus) Rostratum leaf spot, Helminthosporium leaf disease, ear and stalk rot (Setosphaeria rostrata, Helminthosporium rostratum), rusts (Puccinia spp., Physopella spp.), Sclerotium ear rot, Southern blight (Sclerotium rolfsii), seed rot-seedling blight (Bipolaris spp., Helminthosporium spp., Diplodia maydis, Exserohilum spp., Fusarium spp., Gibberella spp., Macrophomina phaseolina, Penicillium spp., Phomopsis spp., Rhizoctonia spp., Sclerotium rolfsii, Spicaria spp.), Selenophoma leaf spot (Selenophoma sp.), sheath rot (Gaeumannomyces graminis), shuck rot (Myrothecium gramineum), silage mold (Monascus spp.), smuts (Ustilago spp., Ustilaginoidea virens, Sphacelotheca reiliana, Sporisorium holci-sorghi), Southern corn leaf blight and stalk rot (Cochliobolus heterostrophus, Bipolaris maydis, Helminthosporium maydis), stalk rots (Cercospora sorghi, Fusarium spp., Nectria haematococca, Mariannaea elegans, Mucor spp., Rhopographus zeae, Spicaria spp.), storage rots (Aspergillus spp., Penicillium spp.), tar spot (Phyllachora maydis, Monographella maydis, Coniothyrium phyllachorae), Trichoderma ear rot and root rot (Trichoderma viride, Trichoderma lignorum, Hypocrea sp.), white ear rot, root and stalk rot (Stenocarpella maydis, Diplodia zeae), yellow leaf blight (Ascochyta ischaemi, Phyllosticta maydis, Mycosphaerella zeae-maydis) and Zonate leaf spot (Gloeocercospora sorghi).

Compound I has a broad range of efficacy as a fungicide. The exact amount of the active material to be applied is dependent not only on the specific active material being applied, but also on the particular action desired, the fungal species to be controlled, and the stage of growth thereof, as well as the part of the plant or other product to be contacted with the compound. Thus, compound I, and formulations containing the same, may not be equally effective at similar concentrations or against the same fungal species.

Compound I is effective in use with plants in a disease-inhibiting and phytologically acceptable amount. The term “disease-inhibiting and phytologically acceptable amount” refers to an amount of a compound that kills or inhibits the plant disease for which control is desired, but is not significantly toxic to the plant. This amount will generally be from about 0.1 to about 1000 ppm (parts per million), with 1 to 500 ppm being preferred. The exact concentration of compound required varies with the fungal disease to be controlled, the type of formulation employed, the method of application, the particular plant species, climate conditions, and the like. A suitable application rate is typically in the range from about 0.10 to about 4 pounds/acre (about 0.01 to 0.45 grams per square meter, g/m²).

Any range or desired value given herein may be extended or altered without losing the effects sought, as is apparent to the skilled person for an understanding of the teachings herein.

### EXAMPLES

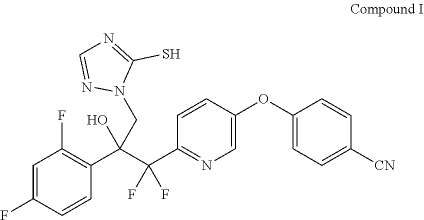 [SMILES: N#Cc1ccc(Oc2ccc(C(F)(F)C(O)(Cn3ncnc3S)c3ccc(F)cc3F)nc2)cc1]

Field Assessment of Puccinia polysora (PUCCPY) in Corn:

A fungicidal treatment containing Compound I, applied alone in an EC formulation at 100 grams of active ingredient per hectare (g ai/ha) and tank mixed with an adjuvant (Agnique BP-420, 50% w/w at 0.3% v/v), was sprayed on corn plants (ZEAMX, P30R50 variety) twice at the growth stages 18 (8 leaves unfolded, application A) and 39 (stem elongation; prior to tassle emergence, application B) at rates of 25, 50, 100, 125 and 150 g ai/ha. The experimental plots were inoculated with brown rust 24 hr after each application. The treatment was part of an experimental trial designed as a randomized complete block with four replications and a plot of approximately 1×3 m. Compound I was applied at water volume of 200 L/ha, using a backpack precision plot sprayer (BKPCKAIR, 2 and 3 m band width, Flat Fan XR110.015 Nozzle) and pressurized at 45 psi.

Disease severity (percentage of visual diseased foliage on whole plot) was assessed six times (15-49 days after application B, DAAB). The disease infection was recorded following EPPO PP1/26 guideline prescriptions. Area under the disease progress curve (AUDPC) was calculated for each plot using the sets of recorded severity data. Relative AUDPC (% control based on AUDPC) was calculated as percent of the nontreated control. Results are given in Table 1.

Field Assessment of Cercospora zeae-Maydis (CERCZM) in Corn:

A fungicidal treatment containing Compound I, applied alone in an EC formulation at 100 grams of active ingredient per hectare (g ai/ha) and tank mixed with an adjuvant (Agnique BP-420, 50% w/w at 0.3% v/v), was sprayed on corn plants (ZEAMX, Formula variety) twice at the growth stages 18 (8 leaves unfolded, application A) and 39 (stem elongation; prior to tassle emergence, application B) at rates of 25, 50, 100, 125 and 150 g ai/ha. The experimental plots were inoculated with grey leaf spot 24 hr after each application. The treatment was part of an experimental trial designed as a randomized complete block with four replications and a plot of approximately 1×3 m. Compound I was applied at water volume of 200 L/ha, using a backpack precision plot sprayer (BKPCKAIR, 2 and 3 m band width, Flat Fan XR110.015 Nozzle) and pressurized at 45 psi.

Disease severity (percentage of visual diseased foliage on whole plot) was assessed six times (15-49 days after application B, DAAB). The disease infection was recorded following EPPO PP1/26 guideline prescriptions. Area under the disease progress curve (AUDPC) was calculated for each plot using the sets of recorded severity data. Relative AUDPC (% control based on AUDPC) was calculated as percent of the nontreated control. Results are given in Table 1.

Field Assessment of Phaeosphaeria maydis (PHSPMA) in Corn:

A fungicidal treatment containing Compound I, applied alone in an EC formulation at 100 grams of active ingredient per hectare (g ai/ha) and tank mixed with an adjuvant (Agnique BP-420, 50% w/w at 0.3% v/v), was sprayed on corn plants (ZEAMX, P30R50 variety) twice at the growth stages 18 (8 leaves unfolded, application A) and 39 (stem elongation; prior to tassle emergence, application B) at rates of 25, 50, 100, 125 and 150 g ai/ha. The experimental plots were inoculated with phaeosphaeria leaf spot 24 hr after each application. The treatment was part of an experimental trial designed as a randomized complete block with four replications and a plot of approximately 1×3 m. Compound I was applied at water volume of 200 L/ha, using a backpack precision plot sprayer (BKPCKAIR, 2 and 3 m band width, Flat Fan XR110.015 Nozzle) and pressurized at 45 psi.

Disease severity (percentage of visual diseased foliage on whole plot) was assessed six times (15-49 days after application B, DAAB). The disease infection was recorded following EPPO PP1/26 guideline prescriptions. Area under the disease progress curve (AUDPC) was calculated for each plot using the sets of recorded severity data. Relative AUDPC (% control based on AUDPC) was calculated as percent of the nontreated control. Results are given in Table 1.

Field Assessment of Exserohilum turcicum (SETOTU) in Corn:

A fungicidal treatment containing Compound I, tank mixed with an adjuvant (Agnique BP-420, 50% w/w at 0.3% v/v), was sprayed on corn plants once at the growth stage 51 (beginning tassel emergence) at rates of 50, 75, 100, and 150 g ai/ha. The treatment was part of an experimental trial designed as a randomized complete block with four replications. Compound I was applied at water volume of 140 L/ha, using a backpack precision plot sprayer.

Disease severity was assessed as combined percent visual necrosis on leaves ear-1, ear and ear+1. Severity was assessed twice at 24 DAA1 (days after application 1) and 32 DAA1. Results are given in Table 2.

### In Each Case of Table 1, the Rating Scale of Percent Control Based on AUDPC is as Follows:

| % Control | Rating |
|---|---|
| 76-100 | A |
| 51-75 | B |
| 26-50 | C |
| 1-25 | D |
| Not tested | E |

TABLE 1
Efficacy of Compound I against Three Fungal
Diseases of Corn with or without Adjuvant.^{a}

| | |
|---|---|
| Rate | Disease (% Control) |

| (g ai/ha) | Adjuvant^{b} | PUCCPY^{c} | CERCZM^{d} | PHSPMA^{e} |
|---|---|---|---|---|
| 100 | N | A | C | B |
| 25 | Y | A | B | B |
| 50 | Y | A | B | B |
| 100 | Y | A | B | A |
| 125 | Y | A | A | A |
| 150 | Y | A | A | A |

^{a}Percent control based on Area Under Disease Progression Curve (AUDPC)
^{b}Agnique BP-420 (50% w/w at 0.3% v/v)
^{c}PUCCPY—brown rust of corn, Puccinia polysora
^{d}CERCZM—grey leaf spot of corn, Cercospora zeae-maydis
^{e}PHSPMA—phaeosphaeria leaf spot of corn, Phaeosphaeria maydis disease complex

TABLE 1
Efficacy^{a} of Compound I for the Control of
Exserohilum turcicum (SETOTU) in Corn.^{b}

| Compound | Rate (g ai/ha)^{c} | 24 DAA1^{d} | 32 DAA1 |
|---|---|---|---|
| I | 50 | 46.5 | 45.5 |
| I | 75 | 45.0 | 44.2 |
| I | 100 | 37.2 | 31.6 |
| I | 150 | 37.9 | 33.3 |
| Priaxor | 220 | 41.8 | 44.9 |
| Untreated Control | | 36.1 | 50.4 |

^{a}Combined visual necrosis on leaves ear − 1, ear, and ear + 1
^{b}Tank mixed with Agnique BP-420 (50% w/w at 0.3% v/v)
^{c}grams of active ingredient per hectare
^{d}24 days after application 1 (DAA1)

What is claimed is:

1. A method of controlling fungal diseases in a corn plant that is at risk of being diseased comprising contacting one of the plant and an area adjacent to the plant with a composition including compound I.

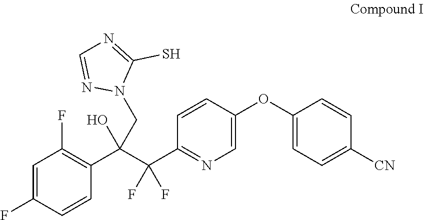 [SMILES: N#Cc1ccc(Oc2ccc(C(F)(F)C(O)(Cn3ncnc3S)c3ccc(F)cc3F)nc2)cc1]

2. The method of claim 1 wherein the composition is

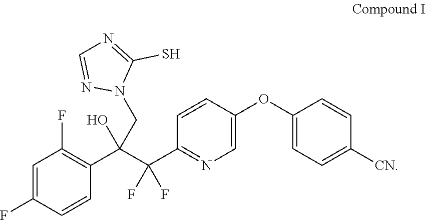 [SMILES: N#Cc1ccc(Oc2ccc(C(F)(F)C(O)(Cn3ncnc3S)c3ccc(F)cc3F)nc2)cc1]

3. The method of claim 1, wherein the composition further includes at least one of one additional agriculturally active ingredient selected from the group consisting of: insecticide, an herbicide, and a fungicide.

4. The method of claim 1, wherein the fungal pathogen is selected from the group consisting of the causal agents of brown rust of corn (Puccinia polysora), grey leaf spot of corn (Cercospora zeae-maydis), phaeosphaeria leaf spot (Phaeosphaeria maydis), and northern corn leaf blight (Exserohilum turcicum).

5. The method of claim 2, wherein the composition further includes at least one of one additional agriculturally active ingredient selected from the group consisting of: insecticide, an herbicide, and a fungicide.

6. The method of claim 2, wherein the fungal pathogen is selected from the group consisting of the causal agents of brown rust of corn (Puccinia polysora), grey leaf spot of corn (Cercospora zeae-maydis), phaeosphaeria leaf spot (Phaeosphaeria maydis), and northern corn leaf blight (Exserohilum turcicum).
